(19) United States
(12) Patent Application Publication
(10) Pub. No.: US 2026/0168951 A1
(43) Pub. Date: Jun. 18, 2026

(54) SENSOR FOR DETECTING VOLATILE BIOGENIC AMINES AND FOOD PACKAGE INCLUDING THE SAME

(71) Applicant: KOC UNIVERSITESI, Istanbul (TR)

(72) Inventors: Emin ISTIF, Istanbul (TR); Levent BEKER, Istanbul (TR); Emel YILGOR, Istanbul (TR); Iskender YILGOR, Istanbul (TR)

(73) Assignee: KOC UNIVERSITESI, Istanbul (TR)

(21) Appl. No.: 19/119,820

(22) PCT Filed: Oct. 10, 2022
(86) PCT No.: PCT/TR2022/051105

§ 371 Date: Apr. 10, 2025

(51) Int. Cl. G01N 27/22; G01N 33/02

(52) U.S. Cl. CPC G01N 27/226; G01N 33/02

(57) ABSTRACT

Embodiments of the present invention relates to a sensor (1) for sensing volatile biogenic amines comprising a substrate (2); an interdigitated electrodes layer (3) on one side of the substrate (2); a copolymer layer (4) comprising maleic anhydride on the interdigitated electrodes layer (3).

### FIELD OF THE INVENTION

The present invention relates to a sensor, especially a battery-free capacitive sensor for real-time sensing volatile biogenic amines to detect food spoilage and a food package comprising the same.

### BACKGROUND OF THE INVENTION

Zero hunger is one of the sustainable development goals to end hunger worldwide. The two crucial phases to achieve this goal are increasing food production and providing safe food to the public, which requires efficient food safety management [1, 2]. Lack of food safety management results in food waste and food-borne disorders such as diarrhea, hemolytic uremic syndrome, and neurological diseases. A plausible solution to provide efficient food safety management and thus prevent food-related ailments is real-time monitoring of the food quality, especially spoilage throughout the food supply chain [3, 4, 5].

One effective way of monitoring spoilage is detecting the volatile biogenic amines (VBAs). Biogenic amines (BAs) are produced by decarboxylation of amide groups in protein-rich foods such as meat, fish, and chicken. A high concentration of BAs in food indicates toxicity and spoilage [6, 7].

There are several approaches to detect the BAs or VBAs for the purpose of monitoring food quality. One of the current practices to detect BAs is based on chromatographic techniques. However, they are not suitable for real-time monitoring since they are expensive, time-consuming and require specific, non-portable equipment and trained personnel.

Another approach is based on visual inspection of VBAs via a colorimetric VBA sensor. The sensor working principle is based on color-changing dyes, inks or nanomaterials observed via a camera system or naked eyes [9, 10, 11, 12, 13, 14, 15]. Since VBA sensors require a high-resolution camera with proper lighting conditions, this approach is not suitable for many applications. Further, colorimetric sensor readings by the naked eye can result in false results if the contrast of the color range for the spoilage indicator is not distinguishable.

Another approach is based on a resistive sensor connected to an inductor. In this approach, the presence of VBAs due to spoilage change the resistivity of the sensor which can be read wirelessly [16, 17, 18]. Although resistive sensor-wireless reader systems are suitable for implementing food packages, such systems are unreliable. The signal amplitude is highly sensitive to the gap between resistive sensor and the reader. The read resistivity may be drastically changed by the distance between the sensor and the portable reader such as a smart phone which results in a false spoilage detection. Thus, resistive sensor-wireless reader systems are prone to motion artifacts.

Due to the known food safety sensors mentioned, there is a need for a sensor, especially a battery-free capacitive sensor for sensing volatile biogenic amines to reliably detect food spoilage and a food package comprising the same in state of the art. The food package with the sensor is able to inform both consumers and supply-chain personnel to classify the food as spoiled or fresh on-demand. Thus, the invention is very effective for monitoring real-time food quality throughout the whole supply chain.

### BRIEF DESCRIPTION OF THE DRAWINGS

An exemplary embodiment of the present invention is illustrated by way of example in the accompanying drawings to be more easily understood and uses thereof will be clearer when considered in view of the detailed description, in which like reference numbers indicate the same or similar elements and the following figures in which:

FIG. 1 is a schematic view of a sensor for sensing volatile biogenic amines in one exemplary embodiment of the present invention.

FIG. 2a is a structural illustration of a copolymer layer in the form of poly(styrene-maleic anhydride) (PSMA) before the reaction with volatile biogenic amines (VBAs) in one exemplary embodiment of the present invention.

FIG. 2b is a graph showing capacitance between interdigitated electrodes when the PSMA copolymer layer does not react with volatile biogenic amines (VBAs) in one exemplary embodiment of the present invention.

FIG. 3a is a structural illustration of a copolymer layer in the form of poly(styrene-maleic anhydride) (PSMA) after the reaction with volatile biogenic amines (VBAs) in one exemplary embodiment of the present invention.

FIG. 3b is a graph showing capacitance change between interdigitated electrodes when the PSMA copolymer layer does and does not react with volatile biogenic amines (VBAs) in one exemplary embodiment of the present invention.

FIG. 4 is a graph showing capacitance change between interdigitated electrodes in the presence of NH3 at different ppms in one exemplary embodiment of the present invention.

FIG. 5 is a graph showing capacitance change between interdigitated electrodes to NH3 and EDA at different ppms in one exemplary embodiment of the present invention.

FIG. 6 is a graph showing capacitance change between interdigitated electrodes in 3 days wherein a chicken sample (about 10 gr) were placed a petri dish together with the sensor at different temperature environment.

FIG. 7 is a graph showing capacitance change between interdigitated electrodes in 3 days wherein a beef sample (about 10 gr) were placed a petri dish together with the sensor at different temperature environment.

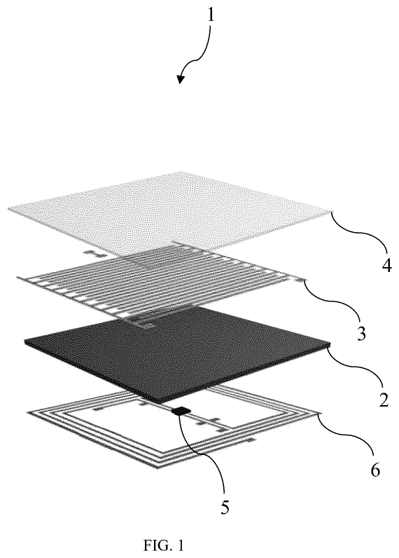

The elements illustrated in the figures are numbered as follows:
1. Sensor
2. Substrate
3. Interdigitated electrodes layer
4. Copolymer layer
5. Silicon component
6. Antenna
MA_B_VBA. Maleic anhydride group of the copolymer before the presence of volatile biogenic amine
MA_A_VBA. Maleic anhydride group of the copolymer after the presence of volatile biogenic amine
B_VBA. Before the presence of volatile biogenic amine
A_VBA. After the presence of volatile biogenic amine
ΔC. Capacitance difference between interdigitated electrodes

### DETAILED DESCRIPTION

Embodiments of the present invention relate to a sensor (1) for sensing volatile biogenic amines comprises a substrate (2); an interdigitated electrode layer (3) on one side of the substrate (2); a copolymer layer (4) comprising maleic anhydride on the interdigitated electrodes layer (3).

One embodiment of the invention comprises a silicon component (5) for capacitance measuring and an antenna (6) for wireless communication. In one alternative of this embodiment, the silicon component (5) and the antenna (6) form an NFC unit such as NFC tag. The silicon component (5) is an NFC chip and the antenna (6) provides inductive coupling with an external data reader with NFC capability such as smart phone. In a variation of this embodiment, the silicon component (5) and the antenna (6) form an RFID unit such as RFID tag. Silicon component (5) is an RFID chip and the antenna (6) provides inductive coupling with an external data reader with RFID such as smart phone.

In one embodiment of the invention, the copolymer layer (4) is poly(styrene-maleic anhydride) (PSMA). PSMA is preferred as copolymer layer (4). One advantage of PSMA is high resistivity for humidity and water which are preferred for food package applications [The monomer styrene(S) and also polystyrene (PS) are not susceptible to neither water nor humidity.]. Another advantage of PSMA is high solubility which makes it easy to be coated on the interdigitated electrodes layer (3). Thus, a sensor (1) which is light such as 2 gr with a small footprint such as 2×2 cm may be produced with ease.

In one embodiment of the invention, the copolymer layer (4) is in a form of

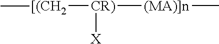

wherein the (MA) stands for maleic anhydride;
R is a hydrogen atom or methyl (CH₃) group;
n is the number of repeat units in PSMA copolymer;
X may be a carboxylic ester group, acetate group, ether group, cyanate group, halogen, or an alkene group. X may be selected from the below groups;

| | | |
|---|---|---|
| COOR₁ | R₁ = CH₃, C₂H₅, C₃H₇, C₄H₉, C₈H₁₇ | carboxylic ester |
| | | group |
| OCOR₂ | R₂ = CH₃, C₂H₅, C₃H₇, C₄H₉, C₈H₁₇ | acetate group |
| OR₃ | R₃ = CH₃, C₂H₅, C₃H₇, C₄H₉, C₆H₁₁, C₈H₁₇ | ether group |
| CN | | cyanate group |
| CH═CH₂ | | alkene group |

The invention works as follows:

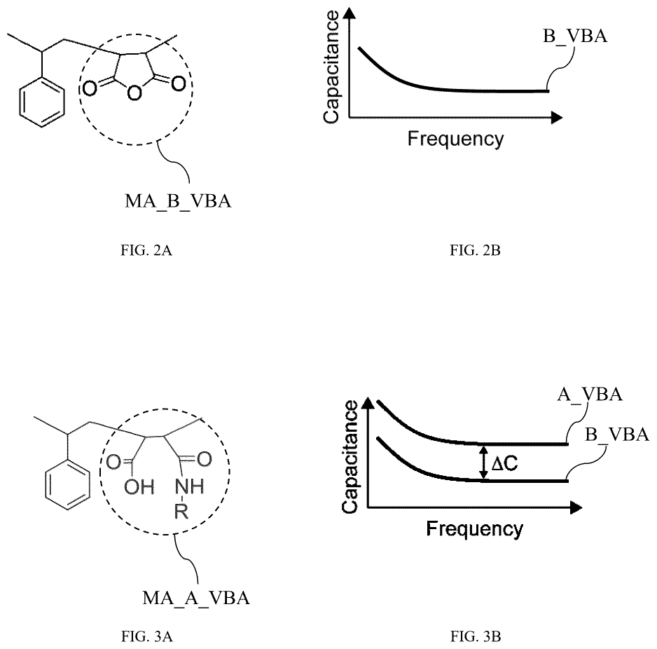

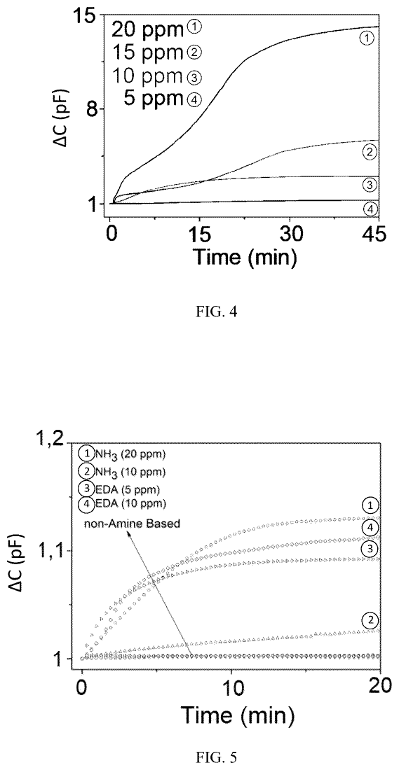

In one embodiment of the invention, the sensor (1) comprises an interdigitated electrodes layer (3) on one side of the substrate (2); a copolymer layer (4) containing maleic anhydride (MA) on the interdigitated electrodes layer (3). The copolymer layer (4) functions as a dielectric layer between the interdigitated electrodes. MA is highly reactive towards volatile biogenic amines (VBAs) which is released from spoiled food. When MA reacts with VBAs, the polarity of the copolymer changes. FIGS. 2a and 3a illustrates the change of capacitance before and after exposing sensor to the VBAs. FIG. 4 depicts ammonia (NH3) sensing of the sensor. The sensor exposed to different concentrations of ammonia for 45 min and capacitance change was observed. Changed polarity of the copolymer alters the dielectric constant of the copolymer. The altered dielectric constant of the copolymer leads to a change in the capacitance value between the interdigitated electrodes. In brief, the chemical reaction between MA and VBAs leads to capacitance change of the sensor (1). Moreover, integrated circuits (ICs) allow smartphones to read the sensor measurement through small-footprint chips. By reading/sensing/detecting/measuring the change of the capacitance value, the presence of the VBAs is recognized which is considered to a food spoilage. Any internal (integrated) or external capacitance reader/sensor can read the capacitance value. The capacitance reader can also include an interface to show the capacitive value, difference or food spoilage state/classification.

In another embodiment of the invention, the sensor (1) further comprises of a silicon component (5) for capacitance measuring and an antenna (6) for wireless communication. Herein, silicon component (5) refers to any kind of capacitance reader/sensor/integrated circuit (IC) which is capable of measuring capacitance between interdigitated electrodes. In this embodiment, silicon component (5) measurements can be transmitted to an external unit which is capable of coupling with the antenna (6) wherein the transmitted measurement data can be visualized by an interface such as a display. Since sensing (1) is based on the capacitance change, the wirelessly transmitted data provides frequency-specific information which is not affected by motion artifacts. In one alternative of this embodiment, the silicon component (5) and the antenna (6) form an NFC unit such as NFC tag. In this alternative, an external data reader with NFC capability, such as a smart phone, is put close to the sensor (1). The silicon component (5) which is an NFC chip, measures the capacitance of the sensor (1) (capacitance value between the interdigitated electrodes). The sensor (1) measurement (capacitance value between the interdigitated electrodes) is transmitted to the external data reader via NFC. In another variation of this embodiment, the silicon component (5) and the antenna (6) form an RFID unit such as an RFID tag. In this alternative, an external data reader with RFID capability such as a smart phone is put close to the sensor (1). The silicon component (5) which is an RFID chip measure the capacitance of the sensor (1) (capacitance value between the interdigitated electrodes). The sensor (1) measurement (capacitance value between the interdigitated electrodes) is transmitted to the external data reader via RFID. For wireless measurement purposes, a mobile phone with NFC capability was used to read the capacitance of the sensor in the presence of VBAs for every second during 20 minutes of experiments. The sensor could detect wirelessly 5 ppm of ethylenediamine (EDA) and 10 ppm of NH3, resulting in a 10% and 3% capacitance change, respectively. (see FIG. 5) Moreover, non-amine-based VOCs did not show any capacitance change confirming the selectivity of the sensor to the volatile primary amines.

In another embodiment of the invention, the sensor (1) comprises a printed circuit board (PCB). This embodiment of the sensor (1) is produced by a printed circuit board (PCB). The substrate (2) is the base of the printed circuit board. FR-4 PCB having a base comprising a glass-fiber reinforced epoxy laminate sheet, and its double side covered with copper may be used. The interdigitated electrodes layer (3) is printed on one side of the printed circuit board (including but not limited to laser printing,). The copolymer layer (4) is coated on the interdigitated electrodes layer (3). The antenna (6) is printed on the other side of the printed circuit board. The silicon component (5) integrated into the printed circuit board. In the preferred alternative of this embodiment, the silicon component (5) is an NFC chip and with antenna (6) forms an NFC unit such as NFC tag.

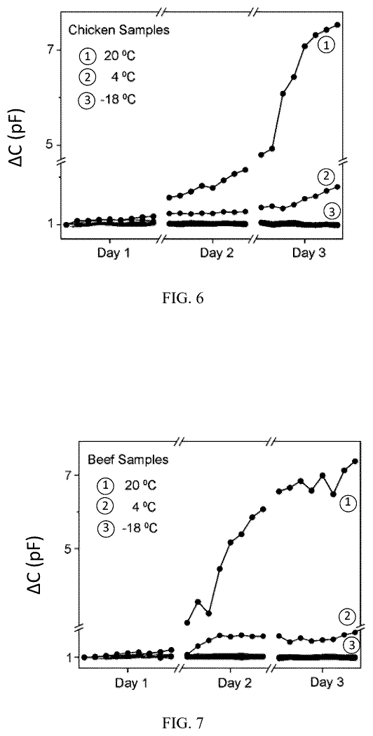

One embodiment of the invention is a food package comprising of a sensor (1) for sensing volatile biogenic amines according to any of the preceding embodiments. In the preferred alternative of this embodiment, the sensor (1) is positioned into the food package such that the sensor (1) does not touch the food but receives the VBAs released from the food. For this alternative, the sensor (1) comprising NFC unit is preferred. By this way, the end user/consumer read the sensor (1) data of the food package via his/her NFC-capable smartphone. The data indicates whether the food is fresh or spoiled which can be displayed on the smartphone. The sensor tested in the presence of chicken and meat samples. The samples were stored in different conditions such as in freezer (−18±4° C.), in a refrigerator (4±4° C.), and room temperature (20±4° C.). The samples were monitored periodically for 8 hours per day for 3 days for each storage condition (FIGS. 3a and 3b). The capacitance of both chicken and beef samples kept at −18±4° C. showed negligible capacitance change. On the other hand, the samples stored at 4±4° C. showed an increasing capacitance (approximately 30% for chicken and 60% for beef) starting from the third day. he samples stored at 20±4° C. shows an increase in capacitance, almost 20% for chicken and beef, starting from the end of the first day. The capacitance shows an increasing trend for both samples stored at room temperature, indicating VBAs released from the spoiled meat. (see FIGS. 6 and 7)

### REFERENCES

[1]. Transforming our world: the 2030 Agenda for Sustainable Development. https://www.un.org/ga/search/view_doc.asp?symbol=A/RES/70/1&Lang=E. United Nations A/RES/70/1 General Assembly (2015)
[2]. United Nations Sustainable Development Goals. Zero Hunger: Why it matters (2020). https://www.un.org/sustainabledevelopment/wp-content/uploads/2016/08/2_Why-It-Matters-2020.pdf
[3]. Lancet Editorial, Food safety really is everyone's business. The Lancet Gastroenterology & Hepatology, 4, 8, 571 (2019).
[4]. Batz, B. B. et al. Long-Term Consequences of food-borne Infections Infect. Dis. Clin. N. Am. 27, 27 599-616 (2013).
[5]. Estimating the burden of food-borne diseases: a practical handbook for countries. Geneva: World Health Organization; 2021. Licence: CC BY-NC-SA 3.0 IGO.
[6]. Spano et al. Biogenic amines in fermented foods. European Journal of Clinical Nutrition, 64, S95 (2010).
[7]. Gardini et al. Technological factors affecting biogenic amine content in foods: A Review. Front. Microbiol. 7, 1218 (2016).
[8]. Suslick, S. K., Li, Z. Portable optoelectronic nose for monitoring meat freshness. ACS Sens. 1, 11, 1330 (2016).
[9]. Wang et al. Fabrication of a “progress bar” colorimetric strip sensor array by dye-mixing method as a potential food freshness indicator. Food Chemistry, 373, 131434 (2022).
[10]. Kim et al. A microneedle technology for sampling and sensing bacteria in the food supply chain. Adv. Func. Mat. 31, 2005370 (2021).
[11]. Andrew Mills. Oxygen indicators and intelligent inks for packaging food. Chem. Soc. Rev. 34, 1003-1011 (2005).
[12]. Chow, F. C. Biogenic amines- and sulfides-responsive gold nanoparticles for real-time visual detection of raw meat, fish, crustaceans, and preserved meat. Food Chemistry, 311, 125908 (2020).
[13]. Andre R. S., Facure, M. H. M., Mercante A. L., Correa, D. S. Electronic nose based on hybrid free-standing nanofibrous mats for meat spoilage monitoring. Sens. Actuators B Chem. 335, 131114 (2022).
[14]. Zhang, Y., Gao, X., Ye, Y., Shen, Y. Fe-Doped polydopamine nanoparticles with peroxidase-mimicking activity for the detection of hypoxanthine related to meat freshness. Analyst (2022).
[15]. Guo et al. Portable Food-Freshness Prediction Platform Based on Colorimetric Barcode Combinatorics and Deep Convolutional Neural Networks. Adv. Mater. 32, 2004805 (2020).
[16]. Barandun et al. Cellulose fibers enable near-zero-cost electrical sensing of water-soluble gases. ACS Sens., 4, 6, 1662 (2019).
[17]. Fiddes et al. Electrochemical detetion of biogenic amines during food spoilage using an integrated sensing RFID tag. Sens. Actuators B Chem. 202, 1298 (2020).
[18]. Ma et al. Highly sensitive, printable nanostructured conductive polymer wireless sensor for food spoilage detection. Nano Lett. 18, 7, 4570 (2018).

1. A sensor (1) for sensing volatile biogenic amines comprising a substrate (2); an interdigitated electrodes layer (3) on one side of the substrate (2); a copolymer layer (4) comprising maleic anhydride on the interdigitated electrodes layer (3).

2. A sensor (1) for sensing volatile biogenic amines according to claim 1 comprises a silicon component (5) for capacitance measuring; an antenna (6) for wireless communication.

3. A sensor (1) for sensing volatile biogenic amines, according to claim 2 or 3, wherein the copolymer layer (4) is poly(styrene-maleic anhydride), wherein the (MA) stands for maleic anhydride; 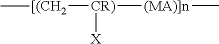
R is a hydrogen atom or methyl (CH₃) group;
n is the number of repeat units in PSMA copolymer;
X may be a carboxylic ester group, acetate group, ether group, cyanate group, halogen, or an alkene group. X may be selected from the below groups;

| | | |
|---|---|---|
| COOR₁ | R₁ = CH₃, C₂H₅, C₃H₇, C₄H₉, C₈H₁₇ | carboxylic ester |
| OCOR₂ | R₂ = CH₃, C₂H₅, C₃H₇, C₄H₉, C₈H₁₇ | acetate group |
| OR₃ | R₃ = CH₃, C₂H₅, C₃H₇, C₄H₉, C₆H₁₁, C₈H₁₇ | ether group |
| CN | | cyanate group |
| CH═CH₂ | | alkene group |

4. A sensor (1) for sensing volatile biogenic amines according to any of the preceding claims, wherein the copolymer layer (4) is in the form of

5. A sensor (1) for sensing volatile biogenic amines according to any of claim 2-4, wherein the silicon component (5) and the antenna (6) forms an NFC unit.

6. A sensor (1) for sensing volatile biogenic amines according to any of claim 2-4, wherein the silicon component (5) and the antenna (6) forms an RFID unit.

7. A sensor (1) for sensing volatile biogenic amines according to any of claim 2-6 comprising of a printed circuit board wherein the substrate (2) is a substrate of the printed circuit board; the interdigitated electrodes layer (3) printed on one side of the printed circuit board; the copolymer layer (4) coated on the interdigitated electrodes layer (3); the antenna (6) printed on the other side of the printed circuit board; the silicon component (5) integrated into the printed circuit board.

8. A food package comprising a sensor (1) for sensing volatile biogenic amines according to any of the preceding claims.
